(19) United States
(12) Patent Application Publication
(10) Pub. No.: US 2025/0154556 A1
(43) Pub. Date: May 15, 2025

(54) SINGLE-MOLECULE RNA STRUCTURE PROFILING

(71) Applicant: JOHN INNES CENTRE, Norwich (GB)

(72) Inventors: Yiliang Ding, Norwich (GB); Minglei Yang, Norwich (GB); Yueying Zhang, Norwich (GB); Jitender Cheema, Norwich (GB)

(21) Appl. No.: 18/840,164

(22) PCT Filed: Feb. 23, 2023
(86) PCT No.: PCT/EP2023/054582

§ 371 Date: Aug. 21, 2024

(30) Foreign Application Priority Data
Feb. 23, 2022 (LU) 501541
Jun. 13, 2022 (GB) 2208604.5

(51) Int. Cl. C12Q 1/6806; C12Q 1/6869

(52) U.S. Cl. CPC C12Q 1/6806; C12Q 1/6869

(57) ABSTRACT

Described is a method for determining structure of an RNA molecule, the method comprising: a) subjecting a population of RNA molecules to structure-specific chemical modifications such that individual RNA molecules are modified; b) reverse-transcribing the modified RNA to provide a complementary DNA (cDNA) molecule, and generating double stranded DNA from said cDNA molecule; c) performing single-molecule sequencing of the double stranded cDNA using a sequencing format which provides multiple reads of each molecule to arrive at a consensus sequence representing a chemical mutation profile for an individual DNA; d) using said chemical mutation profile to determine likelihood of an RNA molecule being single stranded or double stranded at each individual base, to thereby determine the structure of the RNA molecule.

### FIELD OF THE INVENTION

The present invention relates to a method for profiling nucleotide-resolution structure of an RNA molecule at single-molecule level.

### BACKGROUND TO THE INVENTION

The secondary structure of RNA molecules is often critical to performing their biological function (for example, translation, RNA degradation and RNA maturation). Determining secondary structures for RNAs of interest such as viral RNAs or mRNAs is crucial for regulating their corresponding RNA biological process. For instance, understanding the RNA structure of viral RNAs can enable the design of artificial siRNAs or antisense oligos to efficiently target and deactivate viral RNAs via RNA degradation, or the design of small molecules for targeting and inhibiting viral RNA activities such as replication. In addition, RNA structure is known to affect the RNA stability and translation efficiency. Optimizing the RNA structure could increase the translation efficiency and RNA stability, thereby enhancing protein production. Therefore, determining the accurate RNA structure in living cells is important in developing RNA-based gene engineering and therapeutics, e.g., anti-viral siRNAs/antisense oligonucleotides (ASO) and mRNA vaccines.

Recent methods for studying in vivo RNA structure are based on chemical probing. Two main types of chemical reagent can be used. One modifies the Watson-Crick base-pairing face on the nucleobase, as a direct measure of single-strandedness. For instance, Dimethyl sulfate (DMS) is one of the most commonly used nucleobase probing reagents as it easily penetrates the cell, a pre-requisite for in vivo chemical probing¹. The other type of chemical reagent modifies the ribose by selective 2′-hydroxyl acylation and which can be analysed by primer extension (SHAPE)^{2,3}. For instance, NAI (2-methylnicotinic acid imidazolide) is a commonly used SHAPE reagent. Both chemical modifications can be measured by both reverse transcription stopping and mutational profiling⁴⁻⁶. Since 2014, a variety of high-throughput (mostly Illumina-based short-read sequencing) RNA structure chemical probing methods have transformed the scope of RNA structure studies, enabling genome-wide RNA structure analyses⁷. However, there are still three main challenges to obtain accurate RNA structure in vivo:
1) In dissecting the RNA isoform structure heterogeneity: The RNA structure information within the shared regions between isoforms cannot be distinguished by short read sequencing platforms (e.g., Illumina).
2) In determining the RNA structure information for single molecules: Known nanopore-based methods (for example, PORE-cupine) are known, but such methods are unable to achieve true single-molecule resolution of RNA structure information, despite nanopore channels being used to sequence single molecules. The first reason is that the macromolecules in the nanopore channel can be occupied by multiple bases at one time, increasing uncertainty in signal assignment of the nucleotides. Secondly, nanopore-based sequencing has an averaged error rate of 14% for both direct RNA and cDNA sequencing⁸ so cannot give accurate single-molecule information. In order to achieve an acceptable resolution, nanopore-based methods must combine information from multiple single-molecule reads of different individual RNA molecules. Given the inherent variability between individual molecules, this combined information cannot be truly reflective of the structure of RNA at the single-molecule resolution. In contrast, the PacBio platform used in certain examples described herein can achieve 99.9% accuracy at the nucleotide level⁹, facilitating the accurate derivation of RNA structure for each single RNA molecule. The accurate single-molecule read is important in deciphering the RNA structural confirmation diversity at the single-molecule level. This is achieved, in certain embodiments described herein, by use of a sequencing format which provides multiple reads of each molecule to arrive at a consensus sequence representing a chemical mutation profile for an individual nucleic acid molecule.
3) In dissecting the RNA structure conformation heterogeneity: the previous two computational approaches, DREEM¹⁰ and DRACO¹¹ are chemical reactivity-based clustering methods. These methods tend to generate two mutation profiles with extremely high chemical modifications (more single-stranded RNA structure) or extremely low chemical modifications (more double-stranded RNA structure). These clusters directly reflect the similarity of chemical modification efficiencies, but do not directly represent the clusters of RNA structure conformations per se.

We have developed a novel single-molecule structure sequencing method (smStructure-seq) which addresses these challenges by taking advantage of high-accuracy single-molecule sequencing, together with our new analysis pipeline that directly clusters the structural information derived from the mutation profile of each single molecule. This pipeline method (referred to as Determination of the Variation of RNA structure conformation (DaVinci)) incorporates the individual mutation profiles and derives the most-likely RNA structure conformation via a stochastic context-free grammar (SCFG) algorithm independent of thermodynamic parameters. Then the whole conformation space is identified and visualized via PCA analysis. Using the DaVinci method, we could accurately estimate RNA structure conformations at the single-molecule resolution. Therefore, our smStructure-seq allied with DaVinci analysis pipeline can address the challenges of heterogeneities of both isoforms and structural conformations simultaneously and is capable of generating single-molecule RNA structure conformations for each RNA transcript (e.g., isoform). Notably, our smStructure-seq together with our DaVinci analysis pipeline are the first methods which permit resolution of RNA structure information at a true single-molecule level.

### SUMMARY OF THE INVENTION

According to a first aspect of the present invention, there is provided a method for determining structure of an RNA molecule, the method comprising:
a) subjecting a population of RNA molecules to structure-specific chemical treatments where individual RNA molecules will be modified;
b) reverse-transcribing the modified RNA to provide the corresponding complementary DNA (cDNA) molecule, and further generating double stranded DNA from said cDNA molecule;
c) performing single-molecule sequencing of the double stranded cDNA using a sequencing format which provides multiple reads of each molecule to arrive at a consensus sequence representing a chemical mutation profile for an individual DNA;
d) using said chemical mutation profile to determine likelihood of an RNA molecule being single stranded or double stranded at each individual base, to thereby determine the structure of the individual RNA molecule.

This method allows determination of nucleotide-resolution structure of an RNA molecule at a single-molecule level. An individual cDNA molecule is sequenced with multiple reads to determine the consensus sequence representing the chemical mutation profile. This allows accurate determination of the profile for an individual molecule, as variations in the reads will be caused by structure-specific chemical modification in the sequencing process; whereas other methods which combine multiple reads of different molecules—even if having the same nucleotide sequence—cannot control for the possibility of different pairing and/or folding patterns of the original RNA molecules.

By “structure-specific chemical modification” is meant a modification process which is capable of modifying RNA nucleotides based on the single-strandedness of the RNA molecule at that nucleotide. In embodiments the modification process preferentially modifies single-stranded (ie, unpaired) nucleotides. The modification process preferably has no bias towards any particular nucleotides other than paired or unpaired (that is, any unpaired nucleotide is equally likely to be modified regardless of whether it is A, C, G, or U). The modification process may comprise contacting the RNA molecule with a chemical reagent. Two main types of chemical reagent can be used. One modifies the Watson-Crick base-pairing face on the nucleobase, as a direct measure of single-strandedness, e.g, DMS (dimethyl sulfate), kethoxal, glyoxal, EDC (1-ethyl-3-(3-dimethylaminopropyl) carbodiimide), DEPC (diethylpyrocarbonate) and CMCT (1-cyclohexyl-3-(2-morpholinoethyl) carbodiimide metho-p-toluene sulfonate). The other type of chemical reagent is a hydroxyl-selective electrophile, which modifies the ribose by selective 2′-hydroxyl acylation. The hydroxyl-selective electrophile may be selected from 1M7 (1-methyl-7-nitroisatoic anhydride), 1M6 (1-methyl-6-nitroisatoic anhydride), NMIA (N-methylisatoic anhydride), NAI (2-methylnicotinic acid imidazolide), FAI (2-methyl-3-furoic acid imidazolide), and 2A3 (2-aminopyridine-3-carboxylic acid imidazolide). This second category of reagent lends itself to analysis of RNA by primer extension (SHAPE).

During the reverse transcription step, bases which have been chemically modified are more likely to result in mutations than unmodified bases. Suitable reverse transcriptases for use in this step include e.g. murine leukemia virus (M-MLV) RT including Superscript II, Superscript Ill and Superscript IV reverse transcriptase (Invitrogen); avian myeloblastosis virus (AMV); human immunodeficiency virus type 1 (HIV-1) RTs; thermostable Geobacillus stearothermophilus group II intron RT (TGIRT-III); Marathon RT; Maxima H minus; Rocketscript (Bioneer); Thermoscript (Life Technologies); Monsterscript (Illumina) or fidelity (AccuScript; Stratagene); SMARTScribe reverse transcriptases; Induro RT, etc. For example, the reverse transcriptase may be more likely to skip the modified base, or to introduce a mutation into the cDNA strand at that position. When sequenced, an individual cDNA molecule will therefore yield a sequence which may differ from the original RNA sequence in a number of mutated bases. Which bases are mutated will depend on how accessible the original molecule was to the chemical modification, which in turn will depend on the conformation of the RNA. In this way, the consensus sequence generated by the single molecule sequencing can be said to accurately represent a chemical mutation profile for a given individual molecule by identifying which bases are mutated. Comparison of multiple chemical mutation profiles from individual molecules can highlight regions of molecules having the same or similar sequence but differing conformations.

The population of RNA molecules can be subjected to chemical modification in vitro or in vivo. In vivo modification can take place in a living cell, organ, or organism. The cell, organ, or organism may be a plant, fungus, protozoan, prokaryote including bacteria and archea, or animal cell, organ, or organism.

Preferably multiple RNA molecules are subjected to structure-specific chemical modifications; in preferred embodiments of the invention, step a) comprises subjecting a first population of RNA molecules to structure-specific chemical modifications. The RNA molecules of the first population may have identical sequences to one another. This can allow generation of multiple individual chemical mutation profiles, information from which can optionally be combined at or after step d) to determine common regions of likely chemical modification across multiple individual molecules having the same sequence. Alternatively, the RNA molecules of the first population may have similar (but not identical) or different sequences to one another, as discussed below.

In some embodiments, step a) of the method may also comprise subjecting a second population of RNA molecules to structure-specific chemical modifications. Additional further populations (third, fourth, fifth, etc) of RNA molecules may also be included. The RNA molecules of the second population preferably have identical sequences to one another, and a similar (but not identical) or different sequence to those of the first population.

By “having a similar sequence” is meant that the RNA molecules share one or more common regions. In some embodiments the RNA molecules may be variants of one another—for example, having regions of sequence identity and other regions where the sequence differs. In some embodiments, the variant RNA molecules may originate from a common genomic region. Such RNAs may be, for example, isoforms (eg transcription variants or splice variants) from the same gene or coding region; or subgenomic RNAs (eg subgenomic viral RNA). In other embodiments, the variant RNA molecules may originate from duplicated coding regions or members of gene families. Where a first variant RNA is shorter than a second variant RNA, the sequence of the first may be wholly contained within the sequence of the second—that is, there is preferably 100% identity across the length of the shorter RNA. Alternatively, there may be less than 100% identity (eg, 99%, 98%, 97%, 96%, 95%, 94%, 93%, 92%, 91%, 90%, 85%, 80%, 75%, 70% or less identity) over the length of the shorter RNA molecule.

By “having a different sequence” is meant that the RNA molecules do not share common sequences. In some embodiments the RNA molecules are completely dissimilar.

The method is particularly intended to be useful for analysing multiple variant RNA molecules sharing similar sequences which cannot be determined by short read sequencing platforms, as well as for analysing RNA molecules of identical sequence but which may have different structural conformations. Short read sequencing typically cannot sequence fragments of longer than 300 bp, and so when RNA variants share more than this amount of sequence, short read sequencing cannot determine which RNA variant a particular read comes from. Thus, in embodiments, similar RNA molecules share more than 300 nt, preferably more than 400 nt, most preferably more than 600 nt, 750 nt, 1000 nt, 1500 nt, 2000 nt, 2500 nt, 3000 nt, 3500 nt, 4000 nt, 4500 nt, 5000 nt, 10,000 nt, or more than 10,000 nt of common sequence. It is important to note that a population of RNA molecules with the same sequences may contain diverse RNA structural conformations due to the highly dynamic property of RNA molecules. Known methods which use short read sequencing or nanopore sequencing platforms—and which rely on combining information from more than one molecule in order to achieve suitable resolution—cannot resolve the RNA structure information for a single molecule. Therefore, in embodiments, the population of RNA molecules with the same sequences contains diverse RNA structural conformations.

The present method allows multiple RNA molecules of similar sequence to be modified, as well as multiple RNA molecules of the same sequence but which may have different conformations; since the modifications depend on structure of the RNA, different conformations of the RNA molecule will be modified in different ways. These modifications result in incorporation of mutations into the reverse transcribed complementary DNA sequence, and these mutations can be accurately identified by the consensus sequence (eg, a circular consensus sequence, CCS). Multiple consensus sequences can therefore be used to determine whether that site is likely to be single or double stranded in a given conformation—that is, variant RNA molecules with the same sequence can have multiple conformations, each of which will generate a different mutation profile. Combination of information from those unique mutation profiles may therefore indicate the range of different conformations, and the likelihood of a given nucleotide of each single RNA molecule being in a single or double stranded conformation. This can help to elucidate the potential conformation space.

In embodiments, the plurality of RNA molecules may be maintained in different conditions—for example, pH, temperature, light, nutrients, starvation stress, oxidative stress, presence or absence of ions, presence or absence of ions, presence or absence of proteins, presence or absence of metabolites, presence or absence of ligands, presence or absence of chemicals, presence or absence of compounds, presence or absence of pathogens, etc—and the effect of these conditions on single-molecule RNA structure investigated.

In embodiments, the plurality of RNA molecules may be maintained in different cell types—for example, plant cell types (e.g. meristem cell, vascular cell, endodermis cell, etc), animal cell types (e.g. bone cell, sperm cell, fat cell, etc), etc—and the effect of these cell types on single-molecule RNA structure investigated.

In embodiments, the plurality of RNA molecules may be maintained in different developmental stages—for example, plant developmental stages (e.g. vegetative developmental stages and flowering developmental stage, etc) and animal developmental stages (zygote, somitogenesis, organogenesis, etc), etc—and the effect of these developmental stages on single-molecule RNA structure investigated.

In embodiments, the plurality of RNA molecules may be maintained in different species and natural variants—for example, plant species (e.g. Arabidopsis, rice, wheat, barley, soybean, etc), animal species (e.g. zebrafish, Drosophila, mouse, human, worm, etc), etc—and the effect of these organisms and natural variants on single-molecule RNA structure investigated.

In embodiments, the method may further comprise subjecting a control RNA molecule (for example, a molecule which is not subjected to chemical modification) to one or more (and preferably all) of steps b)-d) of the method. The control molecules are used to estimate the experimental noise. In embodiments the control RNA molecule may be part of a control library of molecules; for example, an RNA library obtained from the same source (and hence representing the same mixture of RNA molecules) as the test RNA molecule and/or test RNA library.

The single molecule sequencing method is a method which is capable of obtaining sequence information for single molecules, and is preferably long-read sequencing. By long-read sequencing is meant a method which is capable of sequencing a single molecule of more than 300 nt, preferably more than 400 nt, most preferably more than 600 nt, 750 nt, 1000 nt, 1500 nt, 2000 nt, 2500 nt, 3000 nt, 3500 nt, 4000 nt, 4500 nt, 5000 nt, 10,000 nt, or more than 10,000 nt. In preferred embodiments, the method is designed for use with the PacBio platform for single-molecule real time sequencing. The PacBio platform is generally described in Eid et al, “Real-Time DNA Sequencing from Single Polymerase Molecules”, SCIENCE, 2 Jan. 2009, Vol 323, Issue 5910, pp. 133-138. In brief, double stranded DNAs containing the chemical-induced mutations are ligated to dumbbell adapters to create a circular molecule. A primer and strand-displacement polymerase are introduced, together with labelled nucleotides. Since the molecule is circular, repeated rounds of polymerase replication are undertaken, with incorporation of nucleotides being detected at each step. Multiple reads from a single molecule can then be combined to give one highly accurate consensus sequence.

As will be apparent from the disclosures herein, although the determination of RNA structure by DMS/SHAPE-method is known, the resolution of RNA structure information at single-molecule level has not previously been known.

In embodiments of the invention, the method may be carried out on a library of RNA molecules to determine structures of multiple RNA molecules (whether a sub-population of the whole RNA population, or potentially a transcriptome-wide structurome). For example, the library may be obtained from the RNA population of a cell, tissue, or organ (for instance, an RNA structurome library for a particular tissue). In other embodiments, the library can also include multiple RNA transcripts (e.g., different isoforms) for one or more particular genes; this would allow determination of single-molecule RNA structure conformations for each of the isoforms.

The method may further comprise performing sequence-specific amplification of the cDNA obtained in step b) prior to carrying out step c). This may be of particular benefit where the method is carried out on a library of individual RNAs and allows amplification of a desired target sequence from the whole RNA population. In this way, a given library may be used for determination of different RNA structures for individual genes of interest, or for groups of genes sharing common sequence regions (eg, those arising from gene duplication events, or from differential splicing of RNA), or transcriptomes. The amplification may be thermal amplification, for example, PCR; or may be isothermal amplification, for example, loop-mediated isothermal amplification (LAMP), Helicase-dependent isothermal amplification (HDA), Rolling cycle amplification (RCA), (Recombinase polymerase amplification) RPA, and so on. The skilled person will be aware how to perform such techniques.

In embodiments, the method may further comprise determining RNA structure information at single-molecule level for multiple RNA molecules with the same or different sequences; for example, up to the transcriptome-wide scale.

In embodiments, the step of combining information from multiple mutation profiles may comprise converting each mutation profile to a bit vector to indicate whether a modification occurs at each individual base; and combining multiple bit vectors. The step of combining multiple bit vectors may comprise transforming the bit vectors into single-stranded constraint information—that is, determining for each nucleotide whether it is constrained to be single stranded, or whether base pairing may take place.

In embodiments, the method may further comprise the step of using the structure determined at step d) to generate multiple possible RNA structures for a given molecule, and optionally clustering said multiple possible structures into two or more groups. That is, the determined structure may include probabilities of each base in a given RNA molecule being paired or unpaired, and those probabilities used to generate possible specific structures within the available conformation space. In embodiments, the multiple possible RNA structures are generated independent of thermodynamic parameters; that is, they are based primarily or solely on the A-U and G-C or G-U base-pairing rules. A stochastic context-free grammar (SCFG) may be used to derive the set of possible RNA structures based on the bit-vectors as constraint information. Once generated, the set of possible structures may be clustered by any appropriate algorithm. Where appropriate, clustering may also be carried out directly on the structures determined at step d), without previously generating multiple possible RNA structures for a given molecule. This may be of particular interest when the method is carried out on multiple RNA populations; the determined structures of the populations may be clustered in order to identify particular groups of RNAs (for instance, isoforms) sharing similar regions of conformation space; or when the method is carried out on a population of RNA molecules with the same sequences but diverse RNA structural conformations.

The methods described herein have a number of potential applications based on obtaining accurate RNA structure information. In embodiments, providing the RNA structure information of viral RNAs may enable the design of artificial siRNAs or antisense oligos to efficiently target viral RNAs and deactivate the viral RNAs via RNA degradation. Additionally, it also enables the design of small molecules for targeting the viral RNAs and subsequently inhibiting the viral RNA activities such as replication. Furthermore, this method may further guide the optimization of the RNA structure allowing the enhancements of protein production. The skilled person will be aware of how the structure of the molecule (and accessibility of nucleotides) can impact on these uses.

### BRIEF DESCRIPTION OF THE DRAWINGS

FIG. 1 shows a first workflow for performing methods as described herein

FIG. 2 shows a second workflow for performing methods as described herein

FIG. 3 shows a third workflow for performing methods as described herein

FIG. 4 shows the DaVinci analysis pipeline as described herein

FIG. 5 shows the determined 18S rRNA structure

FIG. 6 shows determined RNA structures for HIV Rev Response Element and pri-miRNA319b

FIG. 7 shows a flowchart of the smStructure-Seq and DaVinci analysis pipeline. The green items are the raw data as the inputs. In section (A), steps of the long read sequencing analysis are highlighted in yellow; and in section (B) steps of the smStructure-Seq Analysis are shown as blue boxes.

FIG. 8 shows the output file COOLAIR3-clusters.png from the workflow given in the Appendix. K-means clustering with the three clusters (k=3) and most representative structures (bit/forgi vector) from each cluster is marked with a red circle.

### DETAILED DESCRIPTION OF THE INVENTION

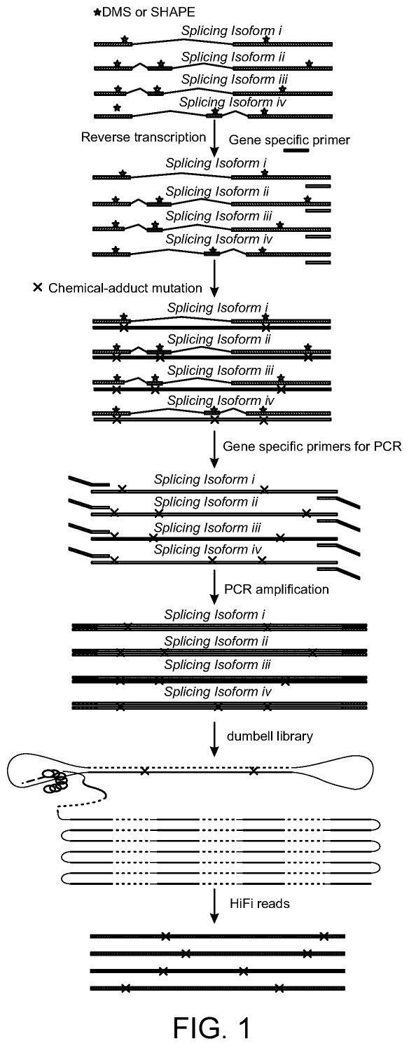

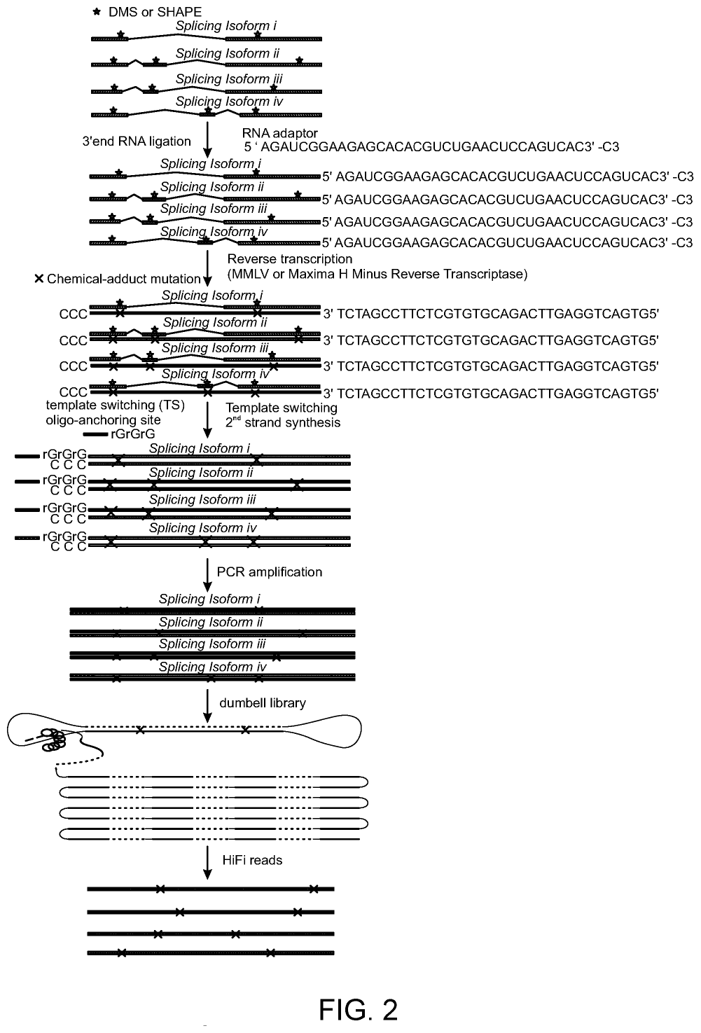

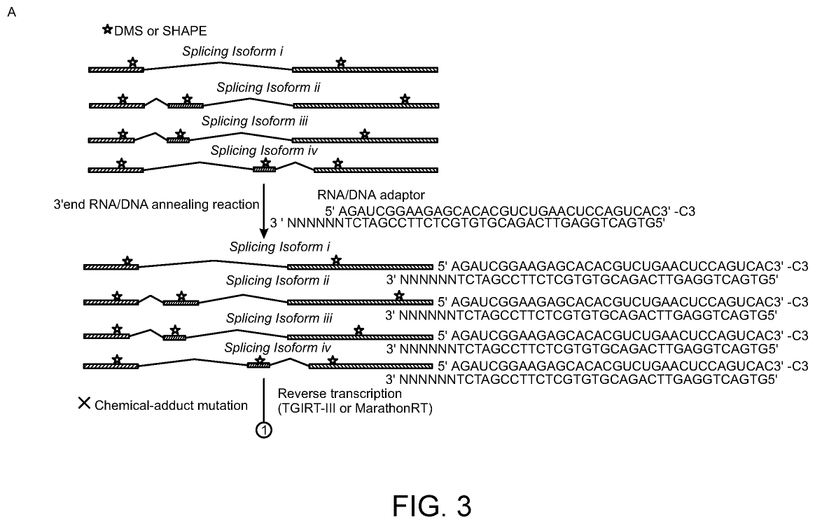

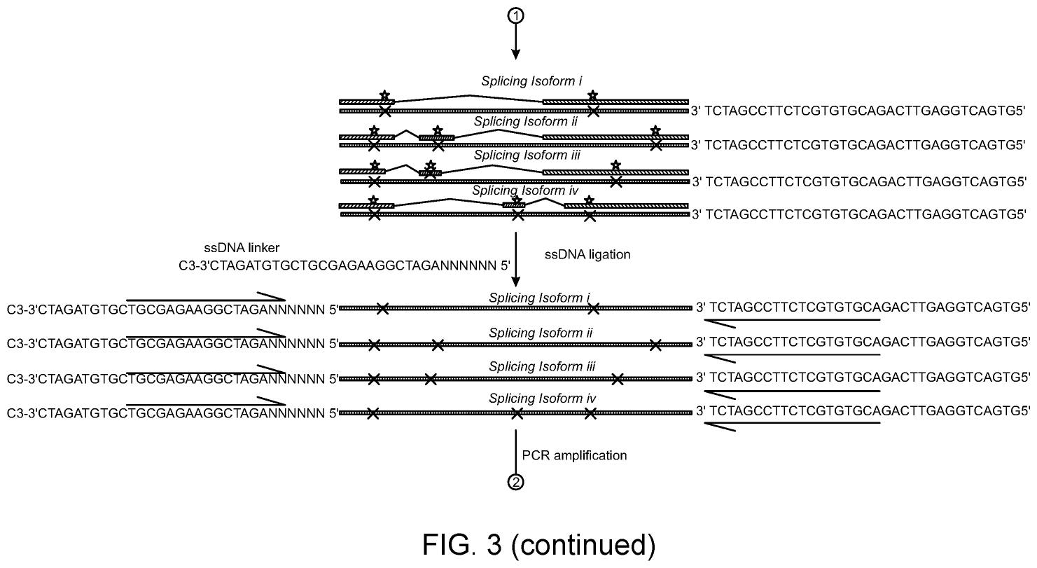

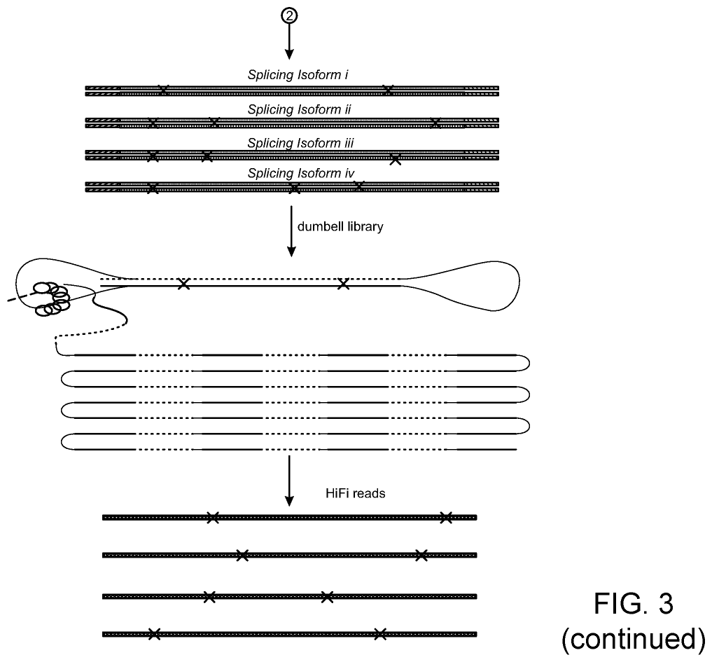

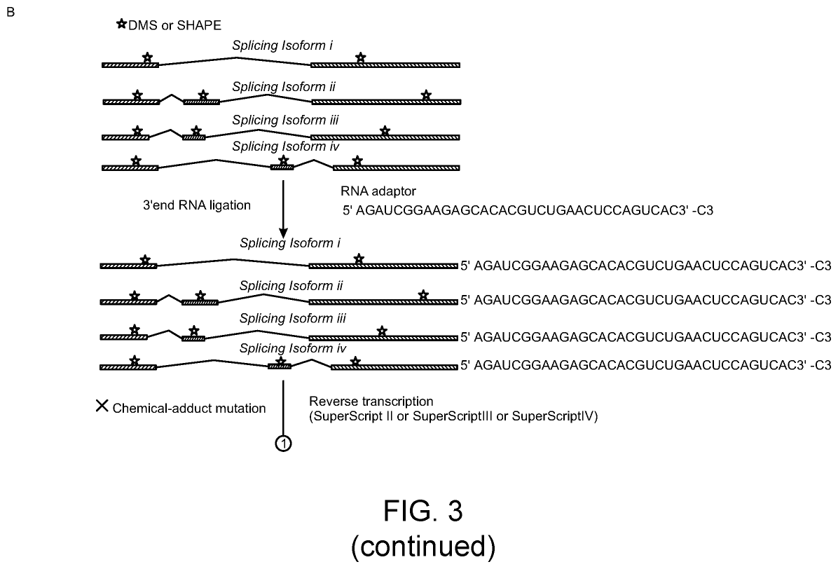

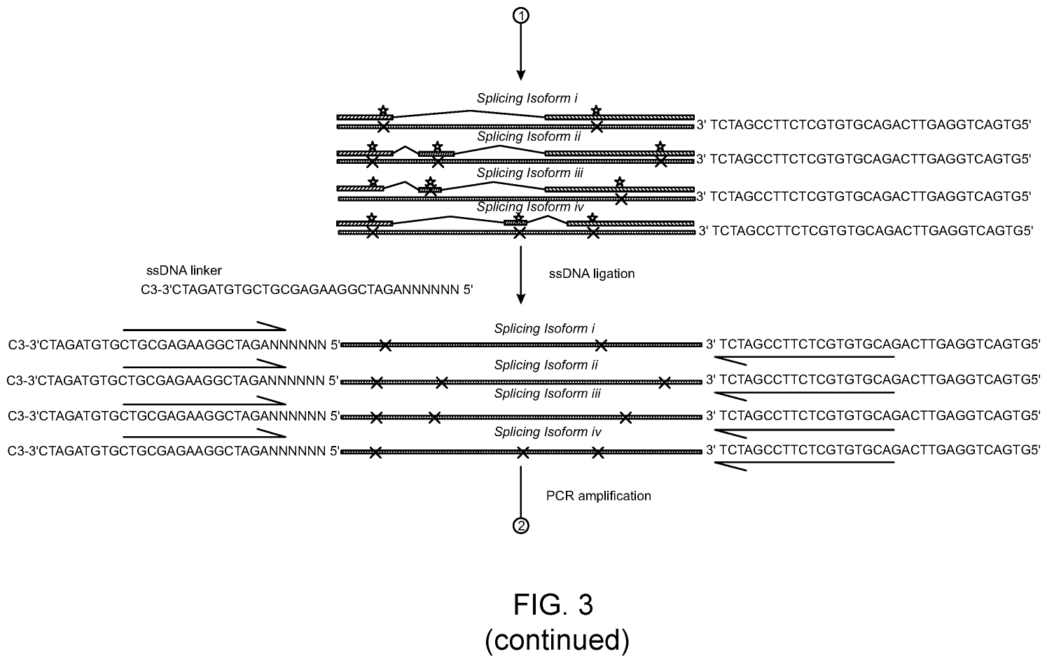

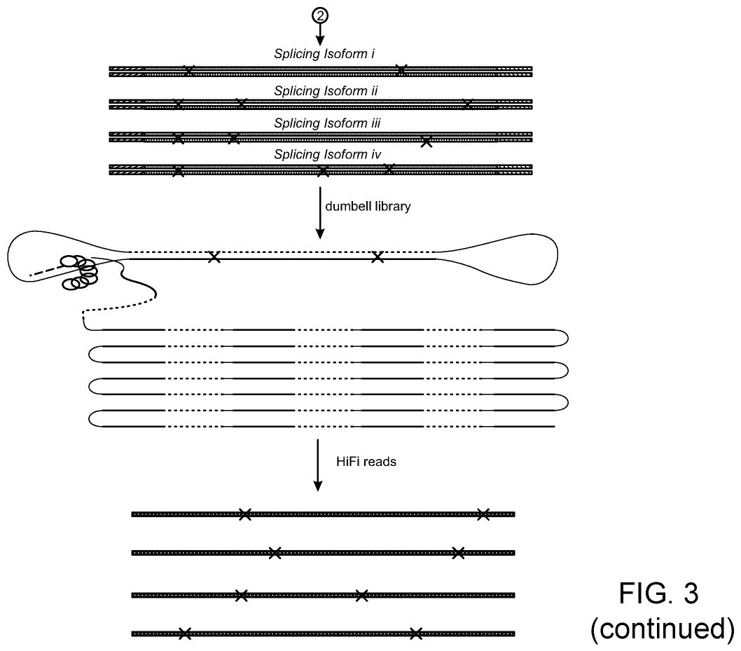

Described herein are three workflows for obtaining RNA structures; each is designed for addressing different aspects of RNA analysis, and hence the skilled person may adopt different workflows for different purposes. The first workflow (FIG. 1) is primarily intended for obtaining the single-molecule RNA structure information of individual genes of interest. The second workflow (FIG. 2) is primarily intended for obtaining the single-molecule RNA structure information of individual long RNAs up to ˜20 kb. The third workflow (FIG. 3) is primarily intended for obtaining the single-molecule RNA structure information of multiple RNAs up to the transcriptome-wide scale.

Methods described herein may also include a new structure analysis pipeline, Determination of the Variation of RNA structure conformation (DaVinci) that incorporates the individual mutation profiles and derives the most-likely RNA structure conformation via a stochastic context-free grammar (SCFG) algorithm independent of thermodynamic parameters. Then the whole conformation space is identified and visualized via PCA analysis. Using the DaVinci method, we could accurately estimate RNA structure conformations at the single-molecule resolution.

Solving In Vivo RNA Structure Conformation at Single Molecule Level (smStructure-Seq)

The RNA structure is an intrinsic property of RNAs which serves an important genetic and functional role of RNA beyond its nucleotide sequence itself carrying coding information. Since 2014, a variety of high-throughput (mostly Illumina-based short-read sequencing) RNA structure profiling methods have transformed the scope of RNA structure studies, enabling genome-wide RNA structure analyses⁷. However, these methods could not resolve RNA structure at the single-molecule level. Our smStructure-seq together with our DaVinci analysis pipeline are the first method that resolve the RNA structure information at the single molecule level, solving three main challenges to decipher the RNA structure in vivo.

The isoform heterogeneity is a major challenge to accurately assign RNA structural information for individual gene-linked isoforms. 90% of human genes¹² and 60% of Arabidopsis genes¹² have alternative splicing events. The RNA structure information within the shared regions between isoforms cannot be distinguished by short read sequencing platforms (e.g., Illumina). Our smStructure-seq addresses this challenge by using the PacBio sequencing method. The sequencing principle of PacBio platform allows the dissection of different transcript isoforms accurately^{9,13-15}, since it requires no assembly step that attenuates the accuracy of isoform assignment.

The second challenge is to determine the RNA structure information for single molecules. RNA structure adopts multiple conformations. Single-molecule structure information as described herein can not only discriminate RNAs with very similar sequence (e.g, isoform or RNA sub-genome in viruses), but can facilitate the identification of RNA structure diversity (the third challenge of RNA structure analysis). Recently, a Nanopore-based method, PORE-cupine¹⁶ was developed to address these challenges. The long-read Nanopore sequencing captures structures along the whole length of each isoform¹⁶. However, the macromolecules in the Nanopore channel can be occupied by multiple bases at one time, increasing uncertainty in signal assignment of the nucleotides^{16,17}. Besides, Nanopore has an averaged error rate of 14% for both direct RNA and cDNA sequencing⁸, which cannot achieve the single-molecular accuracy. In contrast, the long-read, single molecule real time sequencing (eg, PacBio platform) used by smStructure-seq can achieve 99.9% accuracy at the nucleotide level², facilitating the single-molecular accuracy. The accurate single-molecule read is the foundation to deciphering the conformation diversity at the single-molecule level.

The RNA structure can dynamically change in vivo by adopting different conformations. Directly dissecting the heterogeneity of different RNA structural conformations remains challenging. Two computational approaches, DREEM¹⁰ and DRACO¹¹ have been developed. DREEM¹⁰ used an expectation-maximization regime to detect the RNA structure conformation while DRACO¹¹ used an alternative method based on a new clustering regime. These two computational methods were developed to estimate structural conformations based on the Illumina-based platform. Due to the limitation of short read sequencing, the direct dissection of RNA structural conformations has only been achieved for short RNA fragments (200-300 nt)¹⁰ in examples to date, although in theory these methods could be improved for long transcripts. Despite this possibility, these two computational approaches deduce the RNA structure conformation by clustering the chemical reactivity profiles. The chemical reactivity-based clustering methods tend to generate two mutation profiles: one with extreme high chemical modifications (more single-stranded RNA structure) and one with extreme low chemical modifications (more double-stranded RNA structure). These clusters directly reflect the similarity of chemical modification efficiencies rather than directly represent the clusters of RNA structure conformations per se.

Our methods described herein (and referred to as “smStructure-seq”) can solve the challenge by taking advantage of high-accuracy single-molecule sequencing, together with our new analysis pipeline that directly clusters the structural information derived from the mutation profile of each single molecule. This method (named “Determination of the Variation of RNA structure conformation” (DaVinci)), incorporates the individual mutation profiles and derives the most-likely RNA structure conformation via a stochastic context-free grammar (SCFG) algorithm independent of thermodynamic parameters. Then the whole conformation space is identified and visualized via PCA analysis. Using the DaVinci method, we could accurately estimate RNA structure conformations at the single-molecule resolution.

Single-Molecule RNA Structure Profiling (smStructure-Seq)

Our methods described herein (and referred to as “smStructure-seq”) adopt the advantage of long read length and the high accuracy of HiFi reads of the PacBio platform⁷, which is capable of the direct determination of both RNA isoform-specific structures and structural conformations. Our smStructure-seq can address the challenges of heterogeneities of both isoforms and structural conformations simultaneously.

This method is suited to diverse RNA structure probing chemicals such as Dimethyl sulfate (DMS) and 2′-hydroxyl acylation-based chemicals (SHAPE reagent). Extracted RNAs are exposed to the relevant probing chemicals, and subjected to reverse transcription, where the modified sites lead to mutations in the complementary DNA (cDNA). We adapted the resulting cDNAs into the PacBio platform for single-molecule real time sequencing, hence the overall method is termed as single molecule-based RNA structure sequencing (smStructure-seq). The derived raw reads were processed to obtain high-accuracy circular consensus sequences or HiFi reads for generating the RNA structure probing chemical reactivities based on the chemical-adduct mutation profiles.

We have described below three smStructure-seq library construction pipelines, which may be used depending on the properties of different reverse transcriptases. For all pipelines, the initial RNA treatment, and generation of a (+)SHAPE and (−)SHAPE RNA library, is carried out as follows. The example below uses RNA from Arabidopsis thaliana seedlings. Our smStructure-seq is able to be used in other organisms.

(+)SHAPE and (−)SHAPE Single-Molecule Structure-Seq (smStructure-Seq) Library Construction

We used the SHAPE reagent, 2-methylnicotinic acid (NAI) to do the in vivo RNA secondary structure chemical probing. NAI was prepared as reported previously³. Briefly, Arabidopsis thaliana seedlings were completely covered in 20 mL 1×SHAPE reaction buffer (100 mM KCl, 40 mM HEPES (pH7.5) and 0.5 mM MgCl₂) in a 50 mL Falcon tube. NAI was added to a final concentration of 1 M and the tube swirled on a shaker (1000 rpm) for 15 min at room temperature (22° C.) or 30 min at 4° C. This high NAI concentration allows NAI to penetrate plant cells and modify the RNA in vivo. After quenching the reaction with freshly prepared dithiothreitol (DTT), the seedlings were washed with deionized water and immediately frozen with liquid nitrogen and ground into powder. Total RNA was extracted using hot phenol method⁸, followed by DNaseI treatment in accordance with the manufacturer's protocol. The control group was prepared using DMSO (dimethyl sulfoxide, labeled as (−)SHAPE), following the same procedure as described above.

2 μg (+)SHAPE or (−)SHAPE RNA samples was added into 19 μL buffer system containing with 2 μL 0.5 μM RNA-DNA hybrid adaptors (SEQ ID NO 1: 5′rArGrA rUrCrG rGrArA rGrArG rCrArC rArCrG rUrCrU rGrArA rCrUrC rCrArG rUrCrA rC/3SpC3/and SEQ ID NO 2: 5′GTG ACT GGA GTT CAG ACG TGT GCT CTT CCG ATC TN (N=equimolar A, T, G, C)), 4 μL 5× reaction buffer (2.25 M NaCl, 25 mM MgCl₂, 100 mM Tris-HCl, pH 7.5), 2 μL 10×DTT (50 mM; made fresh or from frozen stock), and 1 μL TGIRT®-III enzyme (10 μM; InGex). The reaction system was pre-incubated at room temperature for 30 minutes, then 1 μL of 25 mM dNTPs (an equimolar mixture of dATP, dCTP, dGTP, and dTTP at 25 mM each; RNA grade) added. The whole reaction system in the tube was incubated at 60° C. for 120 minutes. To remove the TGIRT®-III enzyme from the template, 1 μL of 5 M NaOH was added and incubated at 95° C. for 3 minutes. The sample was cooled down to room temperature and neutralized with 1 μL of 5 M HCl before the clean-up of the cDNAs with a MinElute Reaction Cleanup Kit (QIAGEN, Cat. No. 28204). To capture specific target RNAs, PCR reactions with 10 cycles were conducted with primers specific for the desired target using KOD Xtreme™ Hot Start DNA Polymerase (Novagen). Alternatively, non-specific amplification may be used to enrich the library. The amplified DNA fragments from the eight replicates of PCR reactions were merged for achieving sufficient DNAs. The resulting DNAs were size-selected by Solid Phase Reversible Immobilization (SPRI) size selection system (BECKMAN COULTER). Two independent biological replicates were generated for both (+)SHAPE and (−)SHAPE smStructure-seq libraries. The purified DNA samples were subjected to PacBio library construction by BGI company using PacBio Sequel 3.0.

smStructure-seq Data Analysis

The raw reads from (+)SHAPE and (−)SHAPE libraries were converted into HiFi reads (circular consensus sequences, i.e., CCS ) by the utility “ccs” (https://github.com/PacificBiosciences/ccs) with parameters ‘--minPasses=3’ in order to achieve ˜99.8% predicted accuracy (Q30)⁹. The HiFi reads were demultiplexed utilizing demultiplex barcoding algorithm Lima version 1.11.0 (https://github.com/pacificbiosciences/barcoding). The derived HiFi reads were mapped to target reference sequences by using BLASR (version 5.3.3)¹⁹ with parameters “--minMatch 10 -m 5 --hitPolicy leftmost”. Each read was converted into a ‘bit vector’. Briefly, each bit vector corresponds to a single read and consists of series of ‘0’s (representing matches) and ‘1’s (mutations representing mismatches and unambiguously aligned deletions)¹⁰. To generate the overall SHAPE reactivity profiles, the mutation rate (mutr) at a given nucleotide is simply the total number of “1”s divided by the total “0”s and “1”s at that location. Raw SHAPE reactivities were then generated for each nucleotide using the following expression,

$$R = \frac{m u t r ( ( + ) SHAPE ) - m u t r ( ( - ) SHAPE )}{1 - m u t r ( ( - ) SHAPE )}$$

where (+)SHAPE corresponds to a NAI treated sample and (−)SHAPE refers to a DMSO treated sample. 1−mutr((−)SHAPE) was the true negative rate representing the specificity at the specific location. The raw SHAPE reactivity (R) mathematically estimates the positive likelihood ratio of SHAPE modification. The raw SHAPE reactivity was normalized to a standard scale that spanned 0 (no reactivity) to ˜1 (high SHAPE reactivity)²⁰ for showing the mutation profiles.

### Structural Conformation Analysis by DaVinci

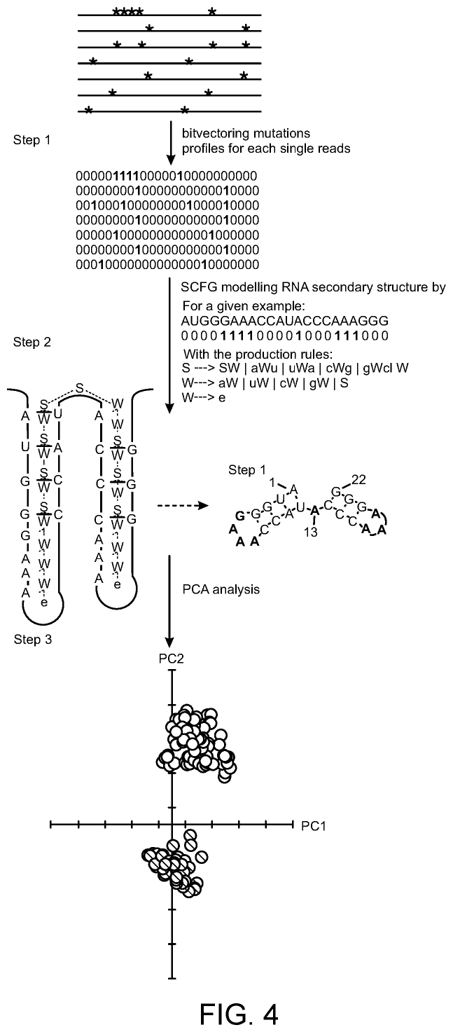

The whole pipeline of Davinci (Determination of the Variation of RNA structure conformation via stochastic context-free grammar) is illustrated in FIG. 4. Each line is referring to one sequencing read. The red stars denote the mutations including mismatch and deletions. The sequencing reads are bit-vectorized following the rules: “0” if a base is wild type and “1” if the base is mutated. SCFG was applied to find the RNA structures that can best-represent each mutation profile. The single-stranded constraints were incorporated into SCFG engine within DaVinci pipeline. The engine of SCF including the set of transformation rules for SCFG and the probability distribution of the transformation rules for each nonterminal symbol, was provided by CONTRAfold²¹ with extended function utility in CentroidFold²² (--engine CONTRAfold -sampling). (Step 2 of FIG. 4 illustrates one possible set of transformation rules which may be used; other potential rules are found in the CONTRAfold package and associated publications, with which the skilled person will be familiar). The generated RNA structures with constraints derived from individual bit vectors were collected. Since different structures can have the same mutation profile during probing, we used the sampling function with constraint of a bit vector to capture multiple structures first. The multiple RNA structures derived from each individual mutation profile were transformed into numeric matrix of RNA structure element (using rnaConvert in the Forgi Package²³) and subjected to Principal Component Analysis (PCA). In all the PCA plots, the x and y axis are the top two component. The results clustered using k-means clustering with the k-means function from the scikit-learn python package²⁴. The value of k was set as determined visually.

The representative structure for each cluster was identified by calculating the most common RNA structure type at each position (i.e., maximum expected accuracy, MEA)²⁵ and then being determined by the RNA structure that is at the center of the whole conformational space and most similar to the most common RNA structure. The base-pair probability (bpp) was calculated by counting the frequency of all present base-pairs in the whole conformation space. The positional base-pair probability was derived by the Pi=Σⱼ^{j}Pij, where Pij is the probability of base i of being base-paired to base j, over all its potential J pairing partners. The likelihood of single-strandedness was calculated by the expression of “1−Pi”. Besides, Shannon entropy is calculated as, Ei=Σⱼ^{j}−Pij*log 10(Pij), where Pij is the probability of base i of being base-paired to base j, over all its potential J pairing partners.

Three smStructure-Seq Pipeline Workflows for Different Applications:

### SmStructure-seq Pipeline Workflow I—FIG. 1

This workflow is for obtaining the single-molecule RNA structure information of individual RNAs of interests. 2 μg DMS or SHAPE treated RNA samples are added into 19 μL buffer system containing 2 μL 0.5 μM gene specific reverse transcription primer, 4 μL 5×reaction buffer (2.25 M NaCl, 25 mM MgCl₂, 100 mM Tris-HCl, pH 7.5), 2 μL 10×DTT (50 mM; made fresh or from frozen stock), and 1 μL SuperScript II or SuperScript Ill or SuperScript IV or TGIRT®-III enzyme or MarathonRT enzyme (additional 1 μL 20 mM MnCl₂). Pre-incubate the reaction system at room temperature for 30 minutes, then add 1 μL of 25 mM dNTPs (an equimolar mixture of dATP, dCTP, dGTP, and dTTP at 25 mM each; RNA grade). The whole reaction system in the tube is incubated at 60° C. for 120 minutes. To remove the reverse transcriptase from the template, 1 μL of 5 M NaOH is added and incubate at 95° C. for 3 minutes. The sample is cooled down to room temperature and neutralized with 1 μL of 5 M HCl before the clean-up of the cDNAs with a MinElute Reaction Cleanup Kit (QIAGEN, Cat. No. 28204). PCR reactions with 10 cycles are conducted with forward and reverse primers containing gene specific primers along with sequences of barcode indexes using KOD Xtreme™ Hot Start DNA Polymerase (Novagen). The amplified DNA fragments from the eight replicates of PCR reactions are merged for achieving sufficient DNAs. The resulting DNAs are size-selected with 1.5% agarose gel, and obtain size fractions (1-2 kb, 2-3 kb, and 3-6 kb) to maximize long reads for capturing all the full-length transcript variants. For individual transcripts with known lengths, the resulting DNAs are size-selected for the specific size range by Solid Phase Reversible Immobilization (SPRI) size selection system (BECKMAN COULTER) following SageELF 2% agarose gel cassette. The purified DNA samples were subjected to PACBIO dumbbell library construction and sequenced using PacBio Sequel I V3.0 or Sequel II.

SmStructure-Seq Pipeline Workflow II (for Long RNAs Up to ˜20 kb)—FIG. 2

This workflow is for obtaining the single-molecule RNA structure information of individual long RNAs up to ˜20 kb. For instance, viral RNAs are usually very long RNAs. This workflow is designed for solving both full-length/subgenome viral RNAs and long RNAs. 2 μg DMS or SHAPE treated RNA samples are added to 10 μL buffer system containing 1 μl 10×T4 PNK buffer, 1 μl T4 PNK enzyme (NEB, M0201L) with no ATP and incubated at 37° C. for 30 min.

1 μl of 50 μM 3′RNA adapter (SEQ ID NO 3: /5rApp/AGAUCGGAAGAGCACACGUCUG/3SpC3/), 1 μl 10×T4 RNA ligase buffer, 6 μl PEG8000, and 2 μl T4 RNA ligase 2 K227Q (NEB, M0351L) were added into the 10 μL sample and incubated at 25° C. for an hour. 1 μL 20 μM DNA primer (SEQ ID NO 4: 5′ GTGACTGGAGTTCAGACGTGTGCTCTTCCGATCT 3′), 1 μL 20 mM MnCl₂, 1 μL 10 mM dNTPs, 4 μL 5×RT reaction buffer, 0.5 μL RiboLock RNase Inhibitor and 1 μL Maxima H Minus enzyme (200 U/μL) are added into ligated RNAs and incubated at 50-65° C. for 180 minutes. Since both MMLV and Maxima H Minus enzyme are capable of adding CCC at the 3′end of cDNA after the first strand cDNA synthesis, then afterwards, the 2^{nd} strand of cDNA is synthesized by SMARTScribe Reverse Transcriptase using template switching (TS) oligo (e.g., SMARTer IIA oligonucleotide) which is a DNA oligo sequence that carries 3 ribo-guanosines (rGrGrG) at its 3′ end. PCR reactions with 10 cycles are conducted with forward and reverse primers using KOD Xtreme™ Hot Start DNA Polymerase (Novagen). The amplified DNA fragments from the eight replicates of PCR reactions are merged for achieving sufficient DNAs. The resulting DNAs are size-selected with 1.5% agarose gel, and obtain three size fractions (1-2 kb, 2-3 kb, and 3-6 kb) to maximize long reads for capturing the full-length transcripts. For individual known transcripts, the resulting DNAs are size-selected for the specific size range by Solid Phase Reversible Immobilization (SPRI) size selection system (BECKMAN COULTER) following SageELF 2% agarose gel cassette. The purified DNA samples were subjected to PACBIO dumbell library construction and sequenced using PacBio Sequel I V3.0 or Sequel II.

### SmStructure-Seq Pipeline Workflow III—FIG. 3

This workflow is for obtaining the single-molecule RNA structure information of multiple RNAs up to the transcriptome-wide scale. 2 μg DMS or SHAPE treated RNA samples are added into 19 μL buffer system containing 2 μL 0.5 μM RNA-DNA hybrid adaptors (SEQ ID NO 1: 5′rArGrA rUrCrG rGrArA rGrArG rCrArC rArCrG rUrCrU rGrArA rCrUrC rCrArG rUrCrA rC/3SpC3/and SEQ ID NO 5: 5′GTG ACT GGA GTT CAG ACG TGT GCT CTT CCG ATC TNNNNNN (N=equimolar A, T, G, C)), 4 μL 5×reaction buffer (2.25 M NaCl, 25 mM MgCl₂, 100 mM Tris-HCl, pH 7.5), 2 μL 10×DTT (50 mM; made fresh or from frozen stock), and 1 μL TGIRT®-III enzyme or MarathonRT enzyme (additional 1 μL 20 mM MnCl₂). Pre-incubate the reaction system at room temperature for 30 minutes, then add 1 μL of 25 mM dNTPs (an equimolar mixture of dATP, dCTP, dGTP, and dTTP at 25 mM each; RNA grade). The whole reaction system in the tube is incubated at 60° C. for 120 minutes. To remove the TGIRT®-III enzyme or MarathonRT enzyme from the template, 1 μL of 5 M NaOH is added and incubate at 95° C. for 3 minutes. The sample is cooled down to room temperature and neutralized with 1 μL of 5 M HCl before the clean-up of the cDNAs with a MinElute Reaction Cleanup Kit (QIAGEN, Cat. No. 28204). ssDNA ligation is performed to attach the ssDNA linker to the 3′end of the cDNA products. There are two ssDNA ligation methods: 1) CircLigase I enzyme ligation with ssDNA linker SEQ ID NO 6: /5Phos/NNNAGATCGGAAGAGCGTCGTGTAG/3SpC3/.

The ssDNA ligation conditions use the CircLigase I enzyme, but they have been modified from the literature and manufacturer's (Epicentre) protocols to optimize ligation. Ligation is performed at 65° C. instead of 60° C., and for 12 h instead of 1 h to facilitate higher ligation yield and to allow time for slower ligating sequences to react. Ligation is followed by heating at 85° C. for 15 min to deactivate the CircLigase I. 2) T4 DNA ligase reaction with ssDNA linker SEQ ID NO 7: /5Phos/AGATCGGAAGAGCGTCGTGTAGCTCTTCCGATCTNNNNNN/3SpC3. In the 20 μL reaction, 10 μl of 2×Quick T4 ligase buffer, 1 μl Quick T4 DNA ligase (NEB, M2200L), and 1 μl 50 M ssDNA linker are added together and incubate at 20° C. overnight. PCR reactions with 10 cycles are conducted with forward and reverse primers (SEQ ID NO 8: 5′ GATCTACACTCTTTCCCTACACGACGCTCTTCCGATCT 3′ and SEQ ID NO 4: 5′ GTGACTGGAGTTCAGACGTGTGCTCTTCCGATCT 3′) using KOD Xtreme™ Hot Start DNA Polymerase (Novagen). The amplified DNA fragments from the eight replicates of PCR reactions are merged for achieving sufficient DNAs. The resulting DNAs are size-selected with 1.5% agarose gel, and obtain three size fractions (1-2 kb, 2-3 kb, and 3-6 kb) to maximize long reads for capturing the full-length transcripts. For individual known transcripts, the resulting DNAs are size-selected for the specific size range by Solid Phase Reversible Immobilization (SPRI) size selection system (BECKMAN COULTER) following SageELF 2% agarose gel cassette. The purified DNA samples were subjected to PACBIO dumbell library construction and sequenced using PacBio Sequel I V3.0 or Sequel II.

In parallel, another pipeline is designed for other reverse transcriptases, e.g., SuperScript II or SuperScript Ill or SuperScript IV. 2 μg DMS or SHAPE treated RNA samples are added to 10 μL buffer system containing with 1 μl 10×T4 PNK buffer, 1 μl T4 PNK enzyme (NEB, M0201L) with no ATP and incubated at 37° C. for 30 min. 1 μl of 50 μM 3′RNA adapter (SEQ ID NO 3: /5rApp/AGAUCGGAAGAGCACACGUCUG/3SpC3/), 1 μl 10×T4 RNA ligase buffer, 6 μl PEG8000, and 2 μl T4 RNA ligase 2 K227Q (NEB, M0351L) were added into the 10 μL sample and incubated at 25° C. for an hour. 1 μL 20 μM DNA primer (SEQ ID NO 4: 5′ GTGACTGGAGTTCAGACGTGTGCTCTTCCGATCT 3′), 1 μL 20 mM MnCl₂, 1 μL 10 mM dNTPs, 4 μL 5×RT reaction buffer, 0.5 μL RiboLock RNase Inhibitor and 1 μL SuperScript II or SuperScript Ill or SuperScript IV enzyme (200 U/μL) are added into ligated RNAs and incubated at 42° C. for 180 minutes. To remove the SuperScript RT enzyme from the template, 1 μL of 5 M NaOH is added and incubate at 95° C. for 3 minutes. The sample is cooled down to room temperature and neutralized with 1 μL of 5 M HCl before the clean-up of the cDNAs with a MinElute Reaction Cleanup Kit (QIAGEN, Cat. No. 28204). ssDNA ligation is performed to attach the ssDNA linker to the 3′end of the cDNA products. There are two ssDNA ligation methods: 1) CircLigase I enzyme ligation with ssDNA linker SEQ ID NO 6: /5Phos/NNNAGATCGGAAGAGCGTCGTGTAG/3SpC3/. The ssDNA ligation conditions use the CircLigase I enzyme, but they have been modified from the literature and manufacturer's (Epicentre) protocols to optimize ligation. Ligation is performed at 65° C. instead of 60° C., and for 12 h instead of 1 h to facilitate higher ligation yield and to allow time for slower ligating sequences to react. Ligation is followed by heating at 85° C. for 15 min to deactivate the CircLigase I. 2) T4 DNA ligase reaction with ssDNA linker SEQ ID NO 7: /5Phos/AGATCGGAAGAGCGTCGTGTAGCTCTTCCGATCTNNNNNN/3SpC3. In the 20 μL reaction, 10 μl of 2×Quick T4 ligase buffer, 1 μl Quick T4 DNA ligase (NEB, M2200L), and 1 μl 50 M ssDNA linker are added together and incubate at 20° C. overnight. PCR reactions with 10 cycles are conducted with forward and reverse primers (SEQ ID NO 8: 5′ GATCTACACTCTTTCCCTACACGACGCTCTTCCGATCT 3′ and SEQ ID NO 4: 5′ GTGACTGGAGTTCAGACGTGTGCTCTTCCGATCT 3′) using KOD Xtreme™ Hot Start DNA Polymerase (Novagen). The amplified DNA fragments from the eight replicates of PCR reactions are merged for achieving sufficient DNAs. The resulting DNAs are size-selected with 1.5% agarose gel, and obtain three size fractions (1-2 kb, 2-3 kb, and 3-6 kb) to maximize long reads for capturing the full-length transcripts. For individual known transcripts, the resulting DNAs are size-selected for the specific size range by Solid Phase Reversible Immobilization (SPRI) size selection system (BECKMAN COULTER) following SageELF 2% agarose gel cassette. The purified DNA samples were subjected to PACBIO dumbell library construction and sequenced using PacBio Sequel I V3.0 or Sequel II.

Once the samples have been prepared and sequenced, the data is analysed in much the same way regardless of pipeline, as described above.

### Examples

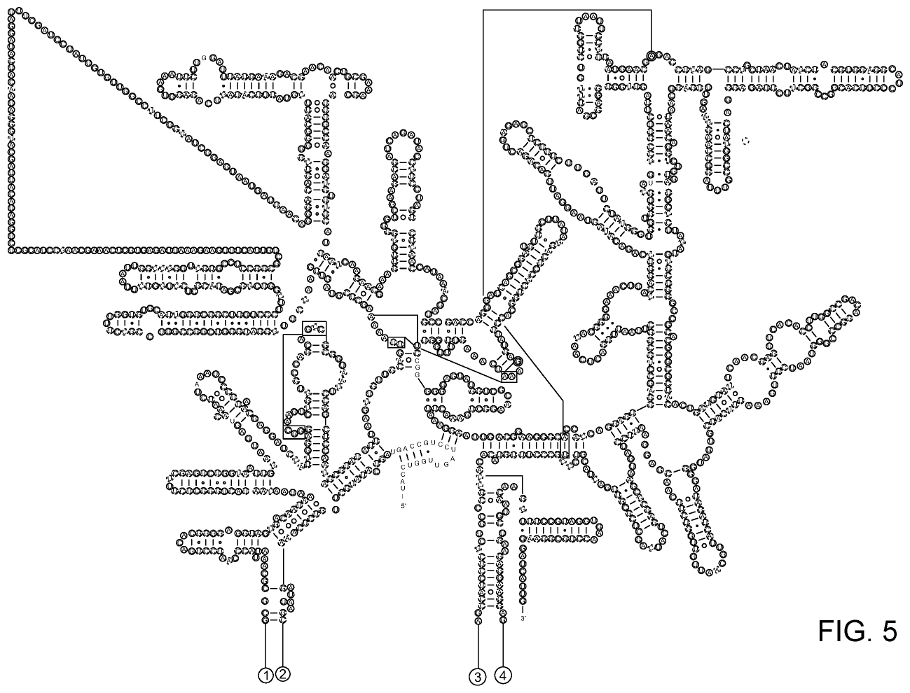

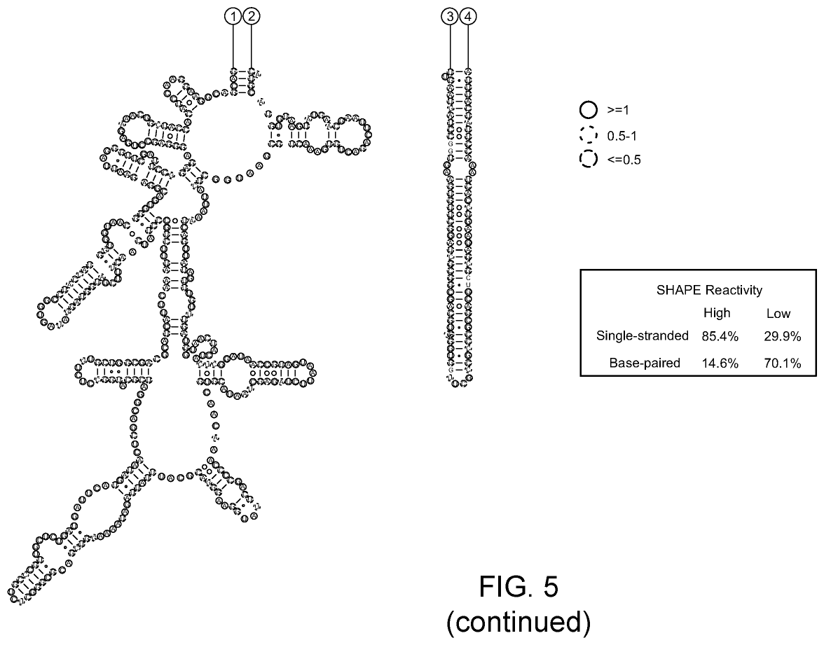

To benchmark the reproducibility and accuracy of our smStructure-seq, we calculated the SHAPE reactivities of 18S rRNA. We found our smStructure-seq libraries were highly reproducible with very high Pearson correlations of 0.95 (P-value <2.2e-16). By comparing our SHAPE reactivities with the 18S rRNA phylogenetic secondary structure²⁶, we found that our smStructure-seq can accurately probe the full-length RNA structure in vivo. We obtained around 8.58 billion total bases (1,317,882 raw reads) of 18S rRNA which serves as the internal control for our smStructure-seq libraries. FIG. 5 shows the complete 18S rRNA (length 1,808 nt) phylogenetic structure is colour-coded according to the SHAPE reactivity generated from our smStructure-seq (SHAPE reactivity >=1 marked in red; SHAPE reactivity 0.5-1 marked in yellow; SHAPE reactivity <=0.5 marked in grey; the primer binding site at the 5′ end was unresolved region and labelled as grey colour). The table quantifies the correspondence between the 18S rRNA phylogenetic structure and the high and low reactivity groups. In the entire 18S rRNA (length=1,808 nt), 85.4% of nucleotides that show high in vivo SHAPE reactivity in our data set correspond to single-stranded regions in the phylogenetic structure (true positive), whereas 70.1% of the nucleotides that show low in vivo SHAPE reactivity correspond to base-paired regions in the phylogenetic structure (true negative). Both true positive (85.4%) and true negative (70.1%) signals are much higher than our previous illumina-based short reads method²⁷.

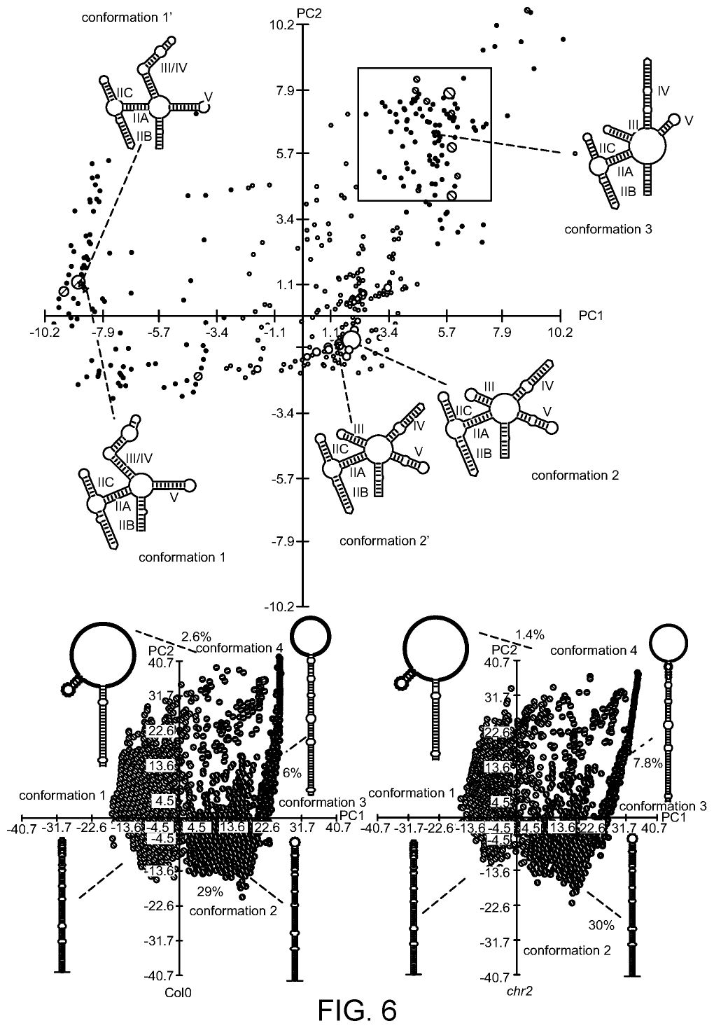

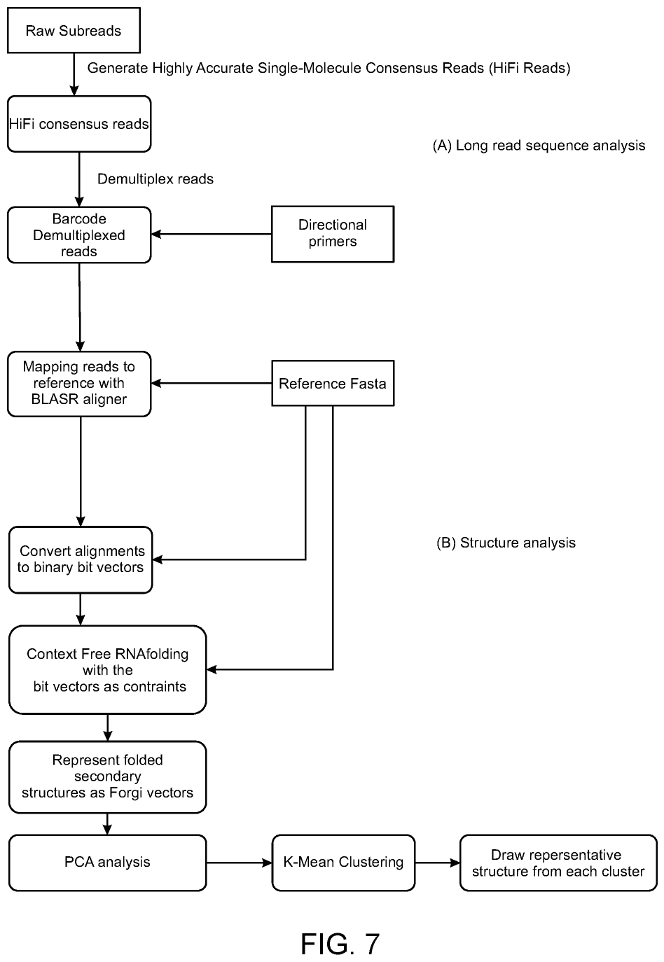

To demonstrate the power of DaVinci, we performed our analysis pipeline on the HIV Rev response element (RRE) that has been reported to be able to adopt alternative conformations that promote different rates of virus replication²⁸. DaVinci was used to analyse the DMS-MaP data of HIV RRE region¹⁰. DREEM used chemical reactivity-based clustering methods and identified two extreme conformations The conformation 1′ and conformation 2′ were the conformation identified by DREEM¹⁰ which are close to the conformations identified by DaVinci (conformation 1 and conformation 2). However, DaVinci could identify an extra cryptic HIV Rev response element (RRE)²⁹ with extended stem IV and short stem V whose conformation was not identified by chemical reactivity-based clustering methods. This conformation (named as RRE61, shown as conformation 3) has been identified to have the ability to confer RevM10 resistance²⁹. See FIG. 6.

A further example comes from analysis of primary-miRNAs RNA secondary structure, important as it affects the efficiency of Microprocessor and Dicing complexes³⁰⁻³² Previous work³³ has shown that a SWI2/SNF2 ATPase CHR2 can change pri-miRNA structures based on the ensemble structure model derived from average chemical modifications. In the chr2-1 mutant, pri-miR319b had a lesser folding or more unpaired nucleotides upper stem, potentially able to affect pri-miRNA processing from the terminal loop to the lower³³. Using our DaVinci analysis pipeline, we revealed at least four conformations in the conformation space of pri-miRNA319b. Rather than changing the ensemble structures, CHR2 changes the populations of individual conformations towards to three conformations with lesser folded upper stem region. These results showed that DaVinci can identify the dynamic nature of in vivo RNA structure conformations, facilitating the investigation of the RNA structural conformation functionality. See FIG. 6, showing the PCA plot and the representative stick RNA structure of pri-miR319b (Left and Right) in Col0 and chr2 mutant.

Therefore, our smStructure-seq allied with DaVinci analysis pipeline can address the challenges of both heterogeneities of isoforms and structural conformations simultaneously and is capable to generate single-molecule RNA structure conformations for each RNA transcript (e.g., isoform).

### Appendix

The following section provides a more detailed walkthrough of the smStructure-Seq analysis pipeline, DaVinci. Reference is made to various publicly-available tools, as well as to additional python scripts written for this purpose. The specific additional python scripts implement the general principles of the method described herein, and are not considered essential to put the present methods and invention into practice, as the skilled person is able to write alternative suitable computer code to implement the invention. The particular additional scripts are mapped-m5-to-bitvectors.py; fold2dotbracketFasta.py; fold-contrafold-uniq-bits-vectors.py; draw-kmeans-clusters.py; run-pca-on-forgi-vectors.py; README-steps.md.

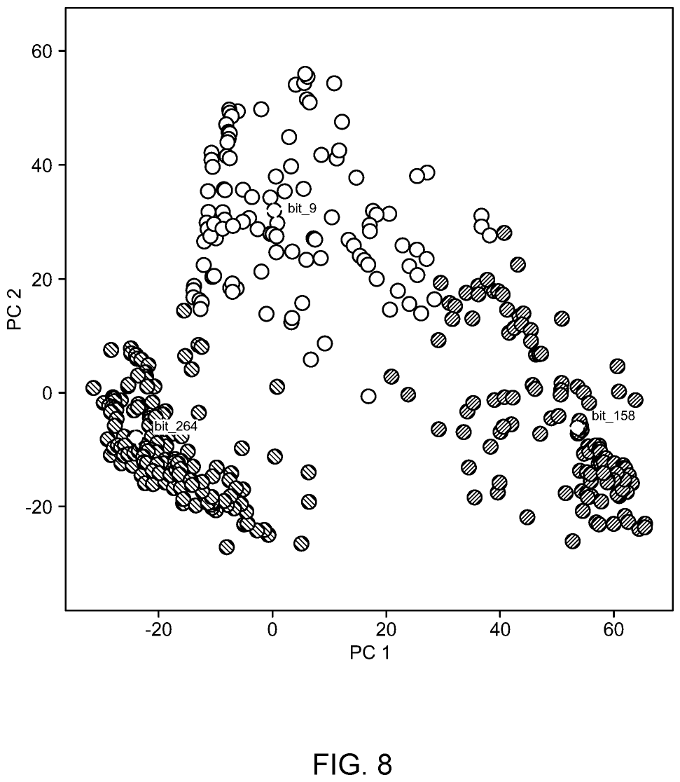

The section also refers to example test data files; these are not given here, but again the skilled person would understand what such example test data files may contain. Similarly, final output files are listed by name. The output file COOLAIR3_R1_R2-clusters.png from this test data is shown in FIG. 8.

smStructure-Seq Analysis Pipeline, DaVinci.
We start with the raw pacbio subreads as the bam and corresponding pbi index files to generate the Single-Molecule Consensus Reads (or HiFi Reads). To generate consensus reads, we used CCS version 4.2.0 (https://github.com/PacificBiosciences/ccs) with minimum 10 passes (Note we have tried different number of passes varying from 3 to 10).
The tools we need for the raw reads analysis namely, pbccs, blasr, lima, bam2fasta were installed through the bioconda channel (https://github.com/bioconda).
Other dependencies, bam2fastx (https://github.com/PacificBiosciences/bam2fastx) and removesmartbell utility from BBTools bioinformatics tools (version BBMap_38.60) (BBMap—Bushnell B.—sourceforge.net/projects/bbmap) were also installed

### There are Two Parts to the Pipeline: (A) Involving Long Read Sequencing Analysis and (B) Single Molecule Structure Analysis:

### (A) Long Reads Sequencing Analysis

Long reads sequencing used the highly accurate sequencing mode to generate single RNA molecule sequence information and the output from the following three steps will be further used for the section (B).

### Step 1: Consensus Generation

Generate Highly Accurate Single-Molecule Consensus Reads (HiFi Reads) using ccs version 4.2.0 (from https://github.com/PacificBiosciences/ccs). We start with the raw pacbio subreads as the bam and corresponding pbi index files to generate the HiFi Reads. We have run the consensus generation with minimum 10 passes (Note we have tried different number of passes varying from 3 to 10).
#ccs 4.2.0 (commit v4.2.0): Generate Highly Accurate Single-Molecule Consensus Re ads (HiFi Reads)
ccs -j 64 --minPasses=10 raw.subreads.bam consensus_reads.ccs2.bam

In this step, we generate a highly accurate single-molecule consensus reads (HiFi Reads). The input to the ccs tool is the raw bam file ‘raw.subreads.bam’ and corresponding output file is the ‘consensus_reads.ccs2.bam’. The parameter ‘minPasses’ should be set to desired rounds of polishing steps; here it is set to a high standard, i.e, 10 rounds. We have computed this on high-performance computing clusters with 64 cores indicated by the parameter (-j 64).

### Step 2: Demultiplex the Barcode

In this step, we demultiplexed the reads HiFi reads by the barcodes. This step is optional if the reads were not multiplexed before the sequencing. We specify the direction primers in a FASTA file by denoting as 5p and 3p for the forward and reverse direction, respectively. Demultiplexed the reads using lima version 1.11.0 (https://github.com/PacificBiosciences/barcoding), a PacBio Barcode Demultiplexer and Primer Remover tool with the following command:
#De-multiplex command
lima --ccs consensus_reads.ccs2.bam primers.fa output.bam --same --split-bam-name d --bam-handles 11 --bam-handles-verbose

In the command above, the primers sequence are stored in the FASTA format file named ‘primers.fa’ and the demultiplexed output bams are given with prefix ‘output.bam’. We used the flag ‘--split-bam-named’ to the names corresponding to the names specified in the primer sequence file. For this case, we set the desired number of bams to ‘--bam-handles 11’, which should be a set number of primers. This step also outputs the corresponding ‘subreadset.xml’ files which allows us the loading of the bam files in the next step.

### Step 3: Convert De-Multiplexed Consensus HiFi Reads to FASTA

We converted the BAM (and the associated xml files) format to the FASTA format for the downstream processes, as conversion allowed us to manually inspect the output. We used bam2fasta (version 1.3.1) from bam2fastx package (https://github.com/PacificBiosciences/bam2fastx) with the following command:
#bam(or.subreadset.xml) to FASTA
bam2fasta -u --output R1_5p output.R1_5p--R1_5p.subreadset.xml
bam2fasta -u --output R2_5p output.R2_5p--R2_5p.subreadset.xml

Here the ‘--output’ is the output name prefix and the input is the xml files which in turns load the bam files for conversion. Note this is an optional step as the bam files can be directly used in the following steps.

### (B) Structure Analysis

Step 1: Mapping of HiFi Reads to the Transcriptome Reference with BLASR Aligner.

We have used the PacBio long read aligner BLASR tool (version 5.1) to map the clean reads to the transcriptome reference (https://github.com/pacificbiosciences/blasr). We set the hitPolicy as leftmost to pick the best hits. We used the m5 format as an output (as this allows for manual inspection of the mapping results).
blasr --hitPolicy leftmost --nproc 8 R1_5p.fasta reference.fasta
--minMatch 10 -m 5 --out R1_5p.m5
blasr --hitPolicy leftmost --nproc 8 R2_5p.fasta reference.fasta
--minMatch 10 -m 5 --out R2_5p.m5

In this step, the input reads for replicate ‘R1_5p.fasta’ and the target reference ‘reference.fasta’ and output file is the R1_5p.m5 which is m5 formatted alignment file. We ran this with a minimum seed length match threshold of 10 with the parameter ‘--minMatch’.

Step 2: Generate Bit Vectors from the Alignment.

The observed mutation profiles for a transcript were converted to a binary vector representation using the bespoke script ‘m5_to_bitvectors.py’. The bit vectors represent sites of mutation and marked as 1 for a mutation(mismatch) or 0 otherwise denoting a wild type (no mutation) state. The region of reference not covered by the reads were marked as ‘NA’.
python3 mapped-m5-to-bitvectors.py --m5_file R1_5p.m5 R2_5p.m5 --transcript COOL AIR3 --reference_file reference.fasta --output_file COOLAIR3_5p.bit

The parameter ‘--transcript’ denotes the name of the target transcript and the ‘-reference_file’ the transcriptome reference in the FASTA format that we used in the mapping step. The input files are a list of m5 formatted files, ‘R1_5p.m5’ and ‘R2_5p.m5’. We are generating a single output bitvector file ‘COOLAIR3_5p.bit’.

### Step 3: Context Free RNA Folding

We use context free RNA folding with contrafold version 2.02 (http://contra.stanford.edu/contrafold/contrafold_v2_02.tar.gz) to get the folded RNA structure files. Then these structure files were further processed through the forgi (https://github.com/ViennaRNA/forgi) tool to get the a numerical representation as Forgi vectors. The script fold-contrafold-uniq-bits-vectors.py folds and generates the Forgi secondary structure representation for the given transcripts. Forgi tool and the associated utility rnaConvert were used (https://github.com/ViennaRNA/forgi).

Corresponding script for the conversion of folded files to the dotbracket format is done via the fold2dotbracketFasta.py script
#Run the contrafold on bitvectors followed by the Forgi
python3 fold-contrafold-uniq-bits-vectors.py --bit_file COOLAIR3_5p.bit --reference_file reference.fasta --transcript COOLAIR3 --size_file bitvector_unique_size.tab --forgi_file forgi-vect-COOLAIR3.txt

### Step 4: PCA Projection

We used the dimension reduction analysis, for example, principal component analysis (PCA) on the Forgi vectors from Scikit-learn: Machine Learning in Python (Pedregosa et al., JMLR 12, pp. 2825-2830, 2011). The projection helps us to view the groupings and separate out the latent classes or to view the conformational space of structures. The following bespoke script gives an output as a png and pdf format along with a csv file for a further manual inspection.
python3 run-pca-on-forgi-vectors.py --input_file forgi-vect-COOLAIR3.txt --tag COOLAIR3-pca --csv_file COOLAIR3-pca.csv

### Step 5: K-means Clustering.

Given a desired number of clusters we perform K-means clustering on the PCA projection using Scikit-learn. We can specify the number of clusters; we have set the default value to three, after trying out different numbers. We visually found three clusters is an optimal number of clusters from the plot.
python3 draw-kmeans-clusters.py --input_file COOLAIR3-pca.csv
--tag COOLAIR3 --num_clusters 3

The parameter ‘--tag’ is the prefix for the output files and the ‘--input_file’ is the csv output file we have from the PCA step. The final outputs will be figures (png and pdf) and a csv file, for this example COOLAIR3-clusters.png, COOLAIR3-clusters.pdf and COOLAIR3-clusters.csv. Clusters 1, 2, and 3 are coloured in the plot as green, orange and pink, respectively.

### Example Test Data

reference.fasta (reference fasta)
primers.fa (primer fasta)
raw.subreads.bam
raw.subreads.bam.pbi
consensus_reads.ccs2.bam
consensus_reads.ccs2.bam.pbi

### Python Scripts:

mapped-m5-to-bitvectors.py
fold-contrafold-uniq-bits-vectors.py
fold2dotbracketFasta.py
run-pca-on-forgi-vectors.py
draw-kmeans-clusters.py

### Final Outputs:

COOLAIR3-clusters.csv
COOLAIR3-clusters.pdf
COOLAIR3-clusters.png

### Other Dependencies:

We used the CCS version 4.2.0 to generate Highly Accurate Single-Molecule Consensus Reads (HiFi Reads) from (https://github.com/PacificBiosciences/ccs)
The tools we need for the raw reads analysis namely, CCS, blasr, lima, bam2fasta were installed through the bioconda channel (https://github.com/bioconda) and
Other dependencies, bam2fastx (https://github.com/PacificBiosciences/bam2fastx) and Fasta parsing utility (https://cithub.com/qitbackspacer/iitu)
Make sure the tools and dependencies are in your PATH.
All the scripts were tested under Python3 (version 3.9.9)

### REFERENCES

1. Yang, X., Yang, M., Deng, H. & Ding, Y. New era of studying RNA secondary structure and its influence on gene regulation in plants. Frontiers in Plant Science 9, 671 (2018).
2. Mortimer, S. A. & Weeks, K. M. Time-resolved RNA SHAPE chemistry. J. Am. Chem. Soc. 130, 16178-16180 (2008).
3. Spitale, R. C. et al. RNA SHAPE analysis in living cells. Nat. Chem. Biol. 9, 18-20 (2013).
4. Siegfried, N. A., Busan, S., Rice, G. M., Nelson, J. A. E. E. & Weeks, K. M. RNA motif discovery by SHAPE and mutational profiling (SHAPE-MaP). Nat. Methods 11, 959-965 (2014).
5. Smola, M. J., Calabrese, J. M. & Weeks, K. M. Detection of RNA-Protein Interactions in Living Cells with SHAPE. Biochemistry 54, 6867-6875 (2015).
6. Zubradt, M. et al. DMS-MaPseq for genome-wide or targeted RNA structure probing in vivo. Nat. Methods 14, 75-82 (2016).
7. Zhang, H. & Ding, Y. Novel insights into the pervasive role of RNA structure in post-transcriptional regulation of gene expression in plants. Biochem. Soc. Trans. 49, 1829-1839 (2021).
8. Workman, R. E. et al. Nanopore native RNA sequencing of a human poly(A) transcriptome. Nat. Methods 16, 1297-1305 (2019).
9. Wenger, A. M. et al. Accurate circular consensus long-read sequencing improves variant detection and assembly of a human genome. Nat. Biotechnol. 37, 1155-1162 (2019).
10. Tomezsko, P. J. et al. Determination of RNA structural diversity and its role in HIV-1 RNA splicing. Nature 582, 438-442 (2020).
11. Morandi, E. et al. Genome-scale deconvolution of RNA structure ensembles. Nat. Methods 18, 249-252 (2021).
12. Pan, Q., Shai, O., Lee, L. J., Frey, B. J. & Blencowe, B. J. Deep surveying of alternative splicing complexity in the human transcriptome by high-throughput sequencing. Nat. Genet. 40, 1413-1415 (2008).
13. Zhao, L. et al. Analysis of transcriptome and epitranscriptome in plants using PacBio Iso-Seq and Nanopore-based direct RNA sequencing. Front. Genet. 10, 1-14 (2019).
14. An, D., Cao, H., Li, C., Humbeck, K. & Wang, W. Isoform sequencing and state-of-art applications for unravelling complexity of plant transcriptomes. Genes (Basel). 9, 43 (2018).
15. Mays, A. D. et al. Single-molecule real-time (SMRT) full-length RNA-sequencing reveals novel and distinct mRNA isoforms in human bone marrow cell subpopulations. Genes (Basel). 10, 253 (2019).
16. Aw, J. G. A. et al. Determination of isoform-specific RNA structure with nanopore long reads. Nat. Biotechnol. 39, 336-346 (2021).
17. Branton, D. et al. The potential and challenges of nanopore sequencing. Nature Biotechnology 26, 1146-1153 (2008).
18. Csorba, T., Questa, J. I., Sun, Q. & Dean, C. Antisense COOLAIR mediates the coordinated switching of chromatin states at FLC during vernalization. Proc. Natl. Acad. Sci. U.S.A 111, 16160-16165 (2014).
19. Chaisson, M. J. & Tesler, G. Mapping single molecule sequencing reads using basic local alignment with successive refinement (BLASR): application and theory. BMC Bioinformatics 13, 238 (2012).
20. Spitale, R. C. et al. Structural imprints in vivo decode RNA regulatory mechanisms. Nature 519, 486-490 (2015).
21. Do, C. B., Woods, D. A. & Batzoglou, S. CONTRAfold: RNA secondary structure prediction without physics-based models. Bioinformatics 22, (2006).
22. Hamada, M., Kiryu, H., Sato, K., Mituyama, T. & Asai, K. Prediction of RNA secondary structure using generalized centroid estimators. Bioinformatics 25, 465-473 (2009).
23. Thiel, B. C., Beckmann, I. K., Kerpedjiev, P. & Hofacker, I. L. 3D based on 2D: Calculating helix angles and stacking patterns using forgi 2.0, an RNA Python library centered on secondary structure elements. F1000Research 8, 287 (2019).
24. Barupal, D. K. et al. Scikit-Learn: Machine Learning in Python. J. Mach. Learn. Res. 12, 2825-2830 (2011).
25. Amman, F. et al. The Trouble with Long-Range Base Pairs in RNA Folding. in Lecture Notes in Computer Science (including subseries Lecture Notes in Artificial Intelligence and Lecture Notes in Bioinformatics) 8213 LNBI, 1-11 (2013).
26. Cannone, J. J. et al. The comparative RNA web (CRW) site: an online database of comparative sequence and structure information for ribosomal, intron, and other RNAs. BMC Bioinformatics 3, 2 (2002).
27. Yang, M. et al. Intact RNA structurome reveals mRNA structure-mediated regulation of miRNA cleavage in vivo. Nucleic Acids Res. 48, 8767-8781 (2020).
28. Sherpa, C., Rausch, J. W., Le Grice, S. F. J., Hammarskjold, M. L. & Rekosh, D. The HIV-1 Rev response element (RRE) adopts alternative conformations that promote different rates of virus replication. Nucleic Acids Res. 43, 4676-4686 (2015).
29. Legiewicz, M. et al. Resistance to RevM10 inhibition reflects a conformational switch in the HIV-1 Rev response element. Proc. Natl. Acad. Sci. U.S.A 105, 14365-14370 (2008).
30. Song, L., Axtell, M. J. & Fedoroff, N. V. RNA secondary structural determinants of miRNA precursor processing in Arabidopsis. Curr. Biol. 20, 37-41 (2010).
31. Werner, S., Wollmann, H., Schneeberger, K. & Weigel, D. Structure determinants for accurate processing of miR172a in Arabidopsis thaliana. Curr. Biol. 20, 42-48 (2010).
32. Mateos, J. L., Bologna, N. G., Chorostecki, U. & Palatnik, J. F. Identification of microRNA processing determinants by rrandom mutagenesis of Arabidopsis MIR172a precursor. Curr. Biol. 20, 49-54 (2010).
33. Wang, Z. et al. SW12/SNF2 ATPase CHR2 remodels pri-miRNAs via Serrate to impede miRNA production. Nature 557, 516-521 (2018).

1. A method for determining structure of an RNA molecule, the method comprising:
a) subjecting a population of RNA molecules to structure-specific chemical modifications such that individual RNA molecules are modified;
b) reverse-transcribing the modified RNA to provide a complementary DNA (cDNA) molecule, and generating double stranded DNA from said cDNA molecule;
c) performing single-molecule sequencing of the double stranded cDNA using a sequencing format which provides multiple reads of each molecule to arrive at a consensus sequence representing a chemical mutation profile for an individual DNA;
d) using said chemical mutation profile to determine likelihood of an RNA molecule being single stranded or double stranded at each individual base, to thereby determine the structure of the RNA molecule.

2. The method of claim 1 wherein the modification process comprises contacting the RNA molecule with a chemical reagent, optionally a hydroxyl-selective electrophile.

3. The method of claim 1 wherein step a) comprises subjecting a first population of RNA molecules to structure-specific chemical modifications.

4. The method of claim 3 wherein the RNA molecules of the first population have identical sequences to one another.

5. The method of claim 3 wherein the RNA molecules of the first population have similar sequences to one another.

6. The method of claim 3 wherein step a) further comprises subjecting a second population of RNA molecules to structure-specific chemical modifications.

7. The method of claim 6 wherein the RNA molecules of the second population have identical sequences to one another, and a similar or different sequence to those of the first population.

8. The method of claim 3 wherein the RNA molecule population(s) originate from a common genomic region.

9. The method of claim 3 wherein the RNA molecules of the or each population share more than 300 nt, preferably more than 400 nt, most preferably more than 600 nt, 750 nt, 1000 nt, 1500 nt, 2000 nt, 2500 nt, 3000 nt, 3500 nt, 4000 nt, 4500 nt, 5000 nt, 10,000 nt, or more than 10,000 nt of common sequence.

10. The method of claim 1 further comprising subjecting a control RNA molecule to one or more, preferably all, of steps b)-d) of the method.

11. The method of claim 10 wherein the control RNA molecule is part of a control library of molecules.

12. The method of claim 1 wherein the single molecule sequencing method is long-read sequencing, and is preferably long read single-molecule real time sequencing.

13. The method of claim 1 wherein the method is carried out on a library of RNA molecules to determine structures of multiple RNA molecules.

14. The method of claim 1 further comprising performing sequence-specific amplification of the cDNA obtained in step b) prior to carrying out step c).

15. The method of claim 1 wherein step d) converting multiple chemical mutation profiles to a bit vector to indicate whether a modification occurs at each individual base; and combining multiple bit vectors.

16. The method of claim 15 wherein the step of combining multiple bit vectors comprises transforming the bit vectors into single-stranded constraint information.

17. The method of claim 1 further comprising the step of using the structure determined at step d) to generate multiple possible RNA structures for a given molecule, and optionally clustering said multiple possible structures into two or more groups.

18. The method of claim 17 wherein the multiple possible RNA structures are generated independent of thermodynamic parameters.

19. The method of claim 1 wherein the RNA molecules are viral RNA molecules.


SEQUENCE LISTING (8 sequences)

SEQ ID NO: 1 - RNA, 34 nt, synthetic construct
  source 1..34: mol_type = other RNA
  modified_base 34: mod_base = OTHER; note = 3' Spacer C3 modification [3SpC3]
agatcggaagagcacacgtctgaactccagtcac

SEQ ID NO: 2 - DNA, 35 bp, synthetic construct
  source 1..35: mol_type = other DNA
gtgactggagttcagacgtgtgctcttccgatctn

SEQ ID NO: 3 - RNA, 22 nt, synthetic construct
  source 1..22: mol_type = other RNA
  modified_base 1: mod_base = OTHER; note = 5' Adenylation (5rApp) modification
  modified_base 22: mod_base = OTHER; note = 3' Spacer C3 Modification [3Sp3C]
agatcggaagagcacacgtctg

SEQ ID NO: 4 - DNA, 34 bp, synthetic construct
  source 1..34: mol_type = other DNA
gtgactggagttcagacgtgtgctcttccgatct

SEQ ID NO: 5 - DNA, 40 bp, synthetic construct
  source 1..40: mol_type = other DNA
gtgactggagttcagacgtgtgctcttccgatctnnnnnn

SEQ ID NO: 6 - DNA, 25 bp, synthetic construct
  source 1..25: mol_type = other DNA
  modified_base 1: mod_base = OTHER; note = 5' Phosphate Oligonucleotide Modification [5'Phos]
  modified_base 25: mod_base = OTHER; note = 3' Spacer C3 Modification [3SpC3]
nnnagatcggaagagcgtcgtgtag

SEQ ID NO: 7 - DNA, 40 bp, synthetic construct
  source 1..40: mol_type = other DNA
  modified_base 1: mod_base = OTHER; note = 5' Phosphate Oligonucleotide Modification [5'Phos]
  modified_base 40: mod_base = OTHER; note = 3' Spacer 3C Modification [3SpC3]
agatcggaagagcgtcgtgtagctcttccgatctnnnnnn

SEQ ID NO: 8 - DNA, 38 bp, synthetic construct
  source 1..38: mol_type = other DNA
gatctacactctttccctacacgacgctcttccgatct